(19) United States
(12) Patent Application Publication
(10) Pub. No.: US 2006/0241647 A1
(43) Pub. Date: Oct. 26, 2006

(54) Hammering inserting rod

(76) Inventor: Shiow-Hui Chen, Hsin Chu (TW)

Correspondence Address: Chen, Shiow-Hui, P.O. Box 166-13, Taipei, 115, TW

(21) Appl. No.: 11/107,722

(22) Filed: Apr. 18, 2005

(51) Int. Cl. A61B 17/58

(52) U.S. Cl. 606/100

(57) ABSTRACT

A hammering inserting rod comprises a tube body; an inserting rod secured to a first end of the tube body; a first end of the inserting rod being a pointed end; a stopper secured to a second end of the tube body; the stopper has a central bore; a linkage passing through the central bore of the stopper; a first end of the linkage being installed with an inner hammer; a hammer secured to a second end of the linkage. By above components, a user grips the hammer to impact the stopper downward continually; or the inner hammer is used to impact against the stopper upward with an upstroke continually; so that the tube body and inserting rod are impelled to anchor into soil or separate from ground.

### BACKGROUND OF THE INVENTION

1. Field of the Invention

The present invention is related to a hammer inserting rod, and particularly to an inserting rod installing such as a big parasol, a flag pole, sign post, a Y type supporter, a camping support rod, plumbing the flagpole, a pole or rod. By means of a hammer preset on top of the inserting rod, impacts against the rod and make it being self anchored into the ground or being separated from the soil.

2. Description of Prior Arts

Generally speaking, an inserting rod is applied to install a big parasol, plumb a flagpole, a pole or a rod; erect a Y-type supporter, and establish a camping support rod. While making use of the inserting rod, the user at least has to bring a hand tool by oneself. For example, a hammer, impacting on the top (i.e. a second end) of the inserting rod leads to the pointed bottom end (i.e. a first end) being anchored into the ground or sandy beach. Therefore, the user has to prepare and carry a hammer accompanied with an umbrella and an inserting rod. It will cause inconveniences for a user to do such preprocessing or pre-production. And the hammer is easy to inflict possible injury on the user or others. Accordingly, a kind of self-help inserting rod, which is characteristic of a hammer fixed on top of the rod, is known to the art. For example, U.S. Pat. No. 6,267,127B1, entitled “self anchoring beach umbrella” issued to Doreste, Eric K. on 31 Jul. 2001. Doreste taught an axial lumen formed within the elongated tubular sleeve, a second end of the tubular sleeve for receiving a pole standard into the axial lumen, an anvil fixed to the pole standard. The user exerts a downward axial force applying and reapplying on the tubular sleeve so that the hammer repeatedly impacting until the pole standard is anchored into the ground and leaving the tubular sleeve in place over the pole standard to hold up an umbrella pole anchored into the ground surface.

The prior arts still has drawbacks as following:
1. After anchoring the inserting rod into the ground, the user is hard to adjust or remove the inserted rod. The pointed first end of the inserting rod has been impelled to anchor into the ground surface, as the first end drills into the ground deeper, the inserting rod is fixed to the soil more securely. It is so hard to pull the inserted rod out of the soil. Without an aid to labor and hand tools, the inserting rod is hard to separate from the soil.
2. The user has to hold the beach umbrella by both hands and exerts a downward axial force repeatedly to drive the tubular sleeve impacting on the hammer and/or anvil, anchors the pole standard into the ground. Because the big parasol or beach umbrella is overweight than a parasol, it sounds like kinds of height discrimination, the users has to be a stout stature for using a big parasol. Younger children or women hardly hold up a big parasol in place over an inserting rod.
3. The top end of the inserting rod has an open end for installing flagpole-like rod, dusts, sands or debris are easily dropped into the open end. It caused extra clean and maintenance work.
4. The tubular sleeve impacting upon the pole standard may cause the tubular sleeve to bend or deform, the vibration accompanied with the impacting causes the stitches between canopy and the ribs become loose and broken.

### SUMMARY OF THE INVENTION

Accordingly, faced the problems of using a prior art inserting rod as mentioned above, the present invention is aimed to provide an inserting rod, which can meet the demands as following: 1. How can the users operate a big parasol over inserting rod without a hand tool (i.e. a hammer), or a workable apparatus (i.e. an umbrella handle) impacting upon the inserting rod. 2. The inserted rod can be pulled out from the hard soil by hand without devoting more muscular strength and endurance to exert axial force onto inserting rod. 3. To prevent dusts, sands and debris from dropping into the open end of the top end of the inserting rod.

To solve the problems as mentioned above, an inserting rod of the present invention comprises:

A tube body, the tube body has an inserting rod secured to its first end, a first end of inserting rod is a pointed end; a stopper fixed to a second end of the tube body, the stopper has a central bore; a linkage passing through the central bore to and fro, the linkage has an inner hammer at its first end; a hammer secured to the second end of the linkage.

To increase the function of the present invention, as the user impacts on the stopper with the hammer installed at a second end of the linkage, it can generate a downward axial force to anchor the tube body and inserting rod into the ground continually. And the user raises the hammer to exert an elevated impact force to the stopper by inner hammer at a first end of the linkage with an upstroke. A linkage between the hammer and inner hammer can move axially relative to the gripping operation of users, and the linkage marked the axially maximum length of the tube body to touch the ground or soil and being subject to the inertia and force applied by the hammer and inner hammer. The tube body has a stopper at its second end. So dusts, sands, and debris are hardly dropped into the second end of the rod as it has a stopper to seal the open end.

Furthermore, the tube body can be applied to tie or bind with a rope, it can be used as a camping support rod, plumbing flagpole, a pole or rod; or others.

The present invention can achieve the merits as following:
1. The inserting rod is anchored into the ground by impacting the hammer upon the stopper downward and the inner hammer hold in mid hollow tube body until lifted up with an upstroke to beat the stopper upward, the hammer and inner hammer are combined with the stopper as a push-pull control system to adjust the inserted rod for anchoring deeper or removing out shallower. It is no need to prepare an extra hand tool to aid the inserting rod.
2. The inserting rod has been equipped with a hammer and an inner hammer, it can be applied to impel the inserting rod from high to low reciprocally, and impacts upon the inserting rod to make it being anchored into the beaching sand or soil. And the inserting rod can be removed, or pulled out from soil, beaching sand easily and freely. Being accustomed to the inner hammer and hammer operation, the users can apply less muscular strength to the inserting rod by exerting an axial force simply in an upward and downward movement. But the user can exert a proper muscular strength to control the inserting or inserted rod to raise or lower as desired with only one hand gripping.
3. Operating the hammer conveniently and easily, either a man or child can operate it. After fixing the hammer and inner hammer to the stopper, it is easy to install the umbrella handle over the inserting rod.
4. A second end of the inserting rod is a stopper sealed end, dusts, sands, and debris hardly being dropped into the sealed end. Keep the inserting rod clean, and avoids from a bothersome clean work afterwards.

### BRIEF DESCRIPTION OF THE DRAWINGS

FIG. 1: an exploded perspective view in the first embodiment of the inserting rod of the present invention;

FIG. 2: an assembly perspective view in the first embodiment of the inserting rod of the present invention;

FIG. 3: a cross sectional view in the first embodiment of the inserting rod of the present invention;

FIG. 4 to 6: schematic views showing the operation of the inserting rod in the first embodiment of the present invention;

FIG. 7 to 11: schematic views showing the inserting rod in the first embodiment of the present invention;

FIG. 12: cross sectional view about the second embodiment of the present invention;

FIG. 13: cross sectional view about the third embodiment of the present invention; and

FIG. 14: schematic view showing the operation of the third embodiment of the present invention.

### DETAILED DESCRIPTION OF THE PREFERRED EMBODIMENTS

The embodiments are described according to the appended drawings hereinafter.

### First Embodiment

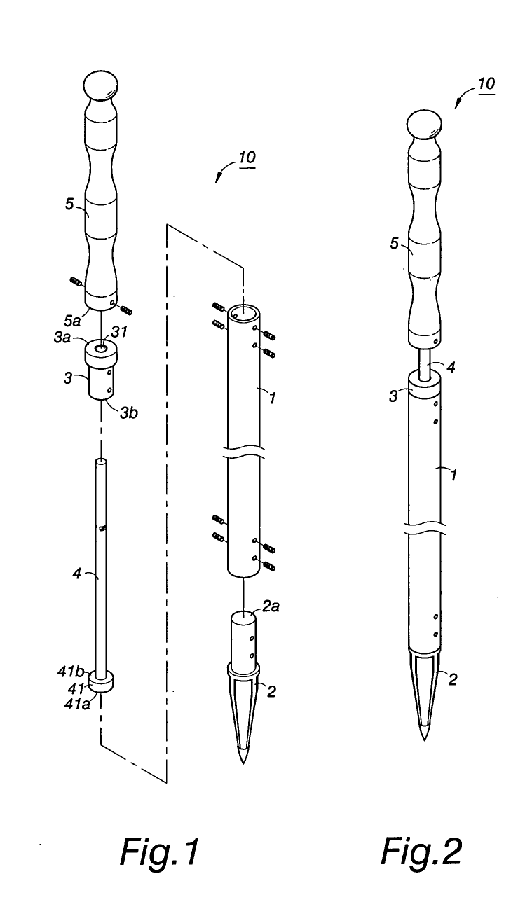

Referring to FIG. 1, a hammering inserting rod of the present invention is illustrated. The hammering inserting rod includes a tube body 1, the tube body 1 has an inserting rod 2 secured to its first end, a stopper 3 fits into a second end of the tube body 1 to seal the second end, the stopper has a central bore, a linkage 4 is designed to pass through the central bore of the stopper 3 to and fro and then entering into the tube body 1; and a hammer 5 is connected to the linkage 4 at a second end of the linkage 4. An assembled perspective view of the hammering inserting rod of the present invention is illustrated in FIG. 2.

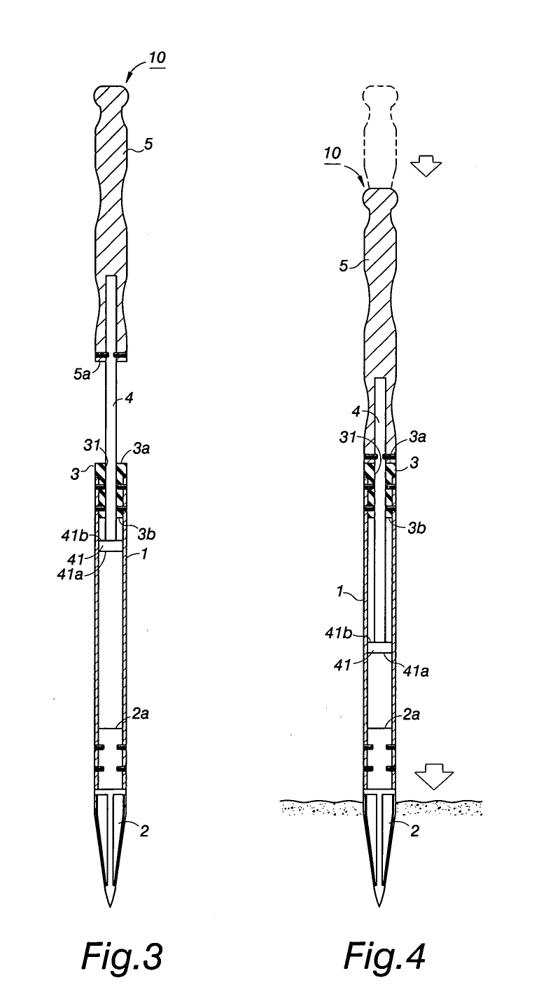

Referring to FIGS. 1 and 3, cross sectional view of the inserting rod according to the present invention is illustrated in FIG. 3. The tube body 1 is a hollow body. The length of the tube body 1 can be determined as desired. The inserting rod 2 is installed at a first end of the tube body 1. The inserting rod 2 can be made individually or integrally with the tube body 1. A first end of the inserting head 2 is a pointed end for being facilitated by anchoring into soil. The stopper 3 has a central bore 31 at its center for being passed by the linkage 4. The stopper 3 fits into an open second end of the tube body 1 and secured to the second end by screws or studs. Or the stopper 3 can be integrally formed with the tube body 1 as a whole one to have the same hinder movement effect and function as mentioned above, i.e., supposed an inner diameter of the second end of the tube body 1 is reduced by forging or punching, it has the same effect as the stopper 3. The linkage 4 is a long rod being passed through the central bore 31 to and fro. The linkage 4 has an inner hammer 41 at its first end. The linkage 4 is connected to the hammer 5 at its second end. The hammer 5 is an exterior hammer set opposite to the inner hammer. The whole shape of the hammer 5 can be lengthened for being held easily and a surface of the hammer may be formed with textures such as linear ring array for preventing from sliding. It is designed according to the principles of ergonomics.

The operation of the present invention will be described herein with reference to FIG. 4. In use, the user lifts the hammer 5 to a certain height suitable for exerting muscular force (referring to FIG. 3). Then an axial force is applied to the hammer 5 to impact. Then a first end 5a thereof impacts on a top surface 3a of a second end of the stopper 3. Thereby, the hammer 5 impacts on the stopper 3 from high to low continually. Once the inserting rod with a pointed first end is anchored a depth into the ground, the user can lift the hammer to a certain height and then impels the hammer again to impact on the stopper. Then the inserting rod 2 will be anchored into the soil deeper and deeper gradually so that the tube body 1 can be firmly secured in the soil easily. Otherwise, supposed the tube body 1 is formed with a shorter length rather than aforesaid tube body. A preset inner hammer 41 installed inside the tube body 1 can be applied to impact upon the top surface 2a of the second end of the inserting rod 2 reciprocally.

The tube body 1 and the inserting rod 2 will withstand the same impacting axial force and enter into soil together. In above two ways of this embodiment, the former one is preferred. Because when the tube body 1 is applied with a force, the inserting rod 2 is also affected. If only the inserting rod 2 suffers a force, the tube body 1 is only affected slightly. Thereby, only to lower the inserting rod has a slowly limited movement without linking the tube body 1.

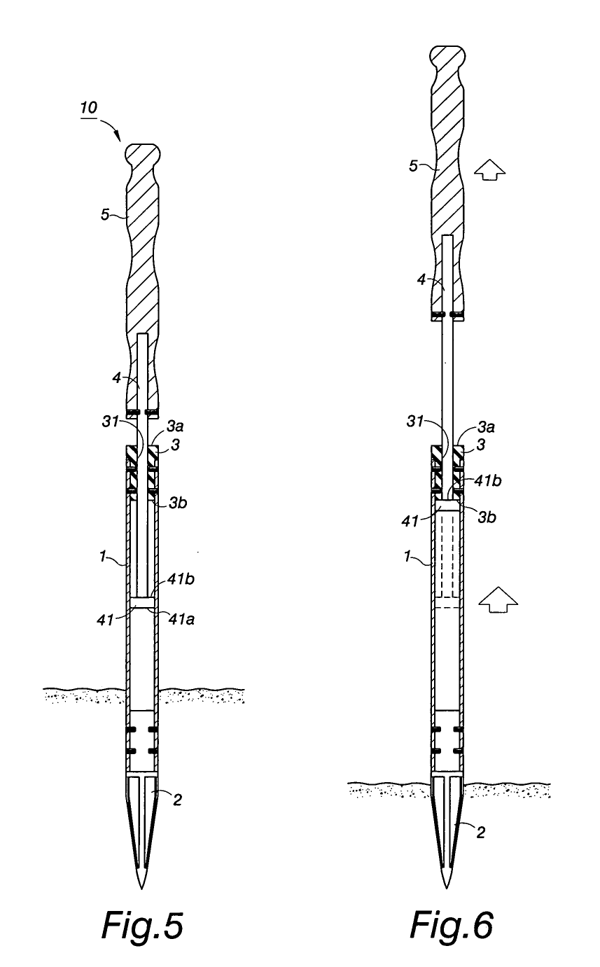

With reference to FIG. 5, if it is desired to separate the inserting rod 10 from the ground, the hammer 5 is lowered to a near bottommost point (i.e. end point) to finish an impact, then lifts the hammer 5 up rapidly so that the top surface 41b of the inner hammer 41 in the tube body 1 impact against a bottom surface 3b of the stopper 3 with an angle of elevation, as shown in FIG. 6. The inner hammer 41 has an upstroke on the stopper 3 once and again, the tube body 1 and inserting rod 2 move upwards and then separate from the ground. Thereby, the inserting rod 10 will leave the ground easily.

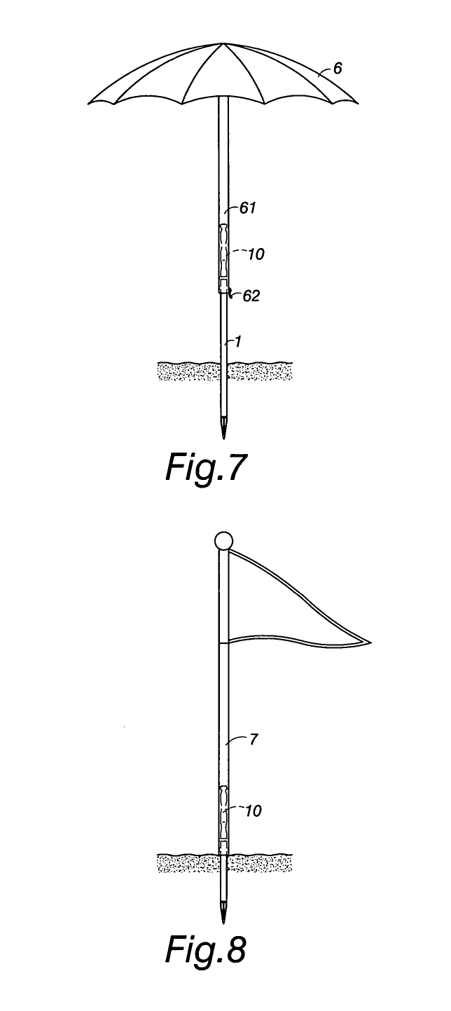

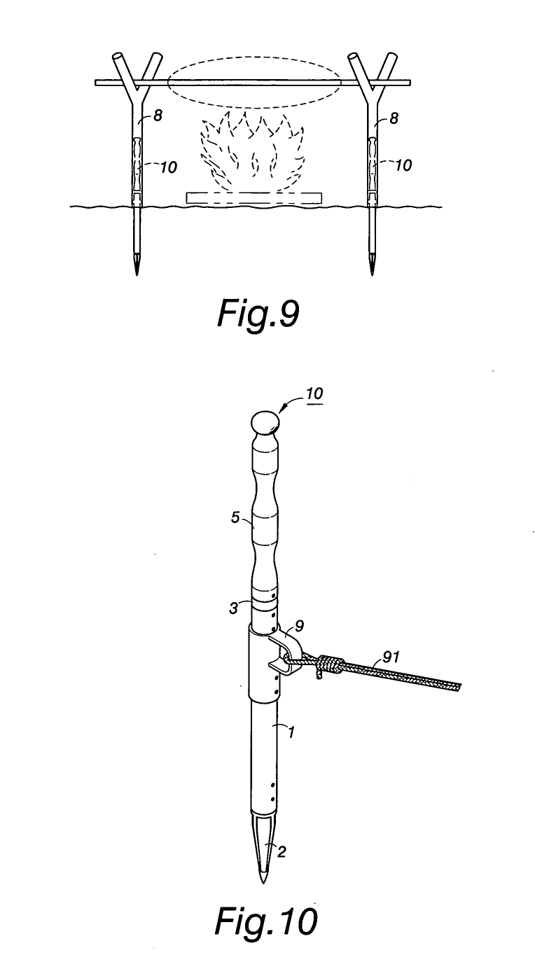

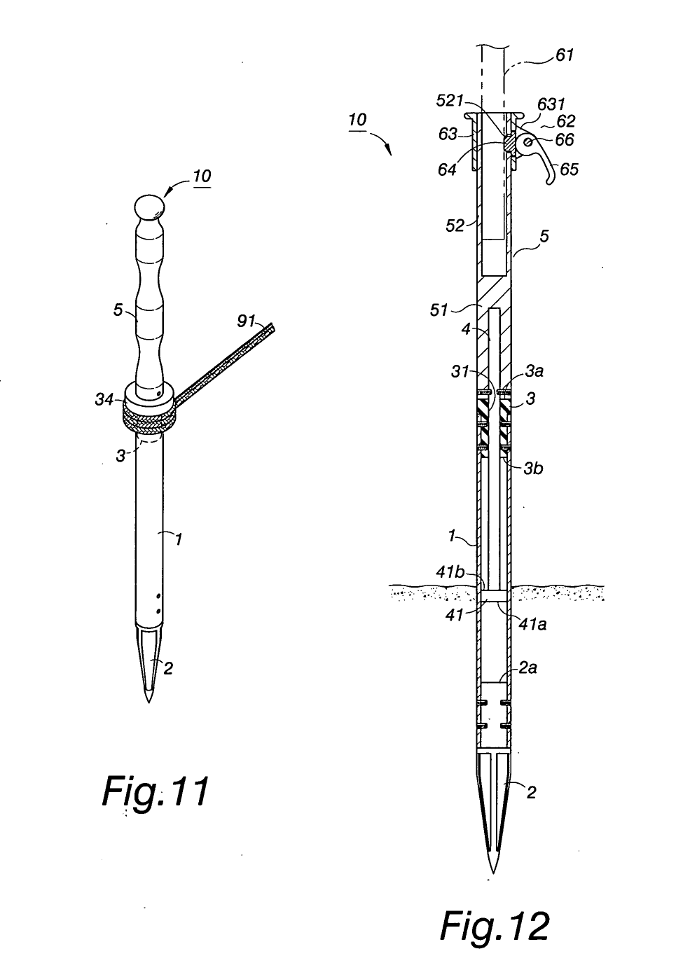

The present invention can be applied to various products. Referring to FIG. 7, a handle 61 of a big parasol 6 can be inserted over an inserting rod 10. That is, the inserting rod 10 is inserted into soil firstly. Then a handle 61 of a big parasol is fastened with the inserting rod 10 by a lever actuated squeezing clamp 62. Moreover, referring to FIG. 8, a hollow flagpole 7 is inserted into the inserting rod 10 in place. Referring to FIG. 9, each of two opposite Y shape supporters 8 is inserted over the inserting rod 10 concurrently. As shown in FIG. 10, an ear 9 (or a through hole) is installed at an outer surface of the tube body 1 of the inserting rod 10 for tying a rope 91 for being used in camping. A top of the tube body 1 may be formed with a flange (not shown) for tying the rope 91. Furthermore, referring to FIG. 11, a rim of a top border of the stopper 3 is expanded to form a bulged rim 34 so that a rope 91 is bound under the flange of the bulged rim 34. Thus can make the rope bound on below the bulged rim 34 of the stopper 3 to protect itself from sliding out and dropping off.

### Second Embodiment

With reference to FIG. 12, the structure of the hammer 5 is changed to form a tube body. A first (i.e. lower) section thereof is a metal hammer 51 whereas a second (i.e. upper) section thereof is a hollow tube 52. A handle 61 of a big parasol 6 can be inserted over the hollow tube 52 and then fastened with the preset lever actuated squeeze clamp 62 installed in the hollow tube 52. The lever actuated squeeze clamp 62 is formed by as following steps: (1.) a sleeve 63 fit over a second section of the hollow tube 52. (2.) A pushing block 64 being disposed on the lateral wall of sleeve 63. (3.) The pushing block 64 can be impelled to pass through a trough hole 521; the trough hole 521 is formed through the lateral wall of a second (i.e. upper) section of the hollow tube body 52. (4.) And a knuckle lever 65 is secured between a pair of protruded ears 631 of the sleeve 63 by a bolt 66; the knuckle lever 521 is used to press down the pushing block 64. In that, the knuckle lever 65 is applied to press down the pushing block 64 after a rotary eccentric operation processed axially through an at least 90-degree arc for optimum movement to its end. Thus can press down the pushing block 64 into the trough hole 521 to clamp or press the handle 61 of a big parasol 6. Thus the handle is secured without separating from a hammer 5. Above mentioned lever actuated squeeze clamp 62 has been used in the prior art. It can be applied to the present invention simply to adapt to the one hand operation more comfortably.

### Third Embodiment

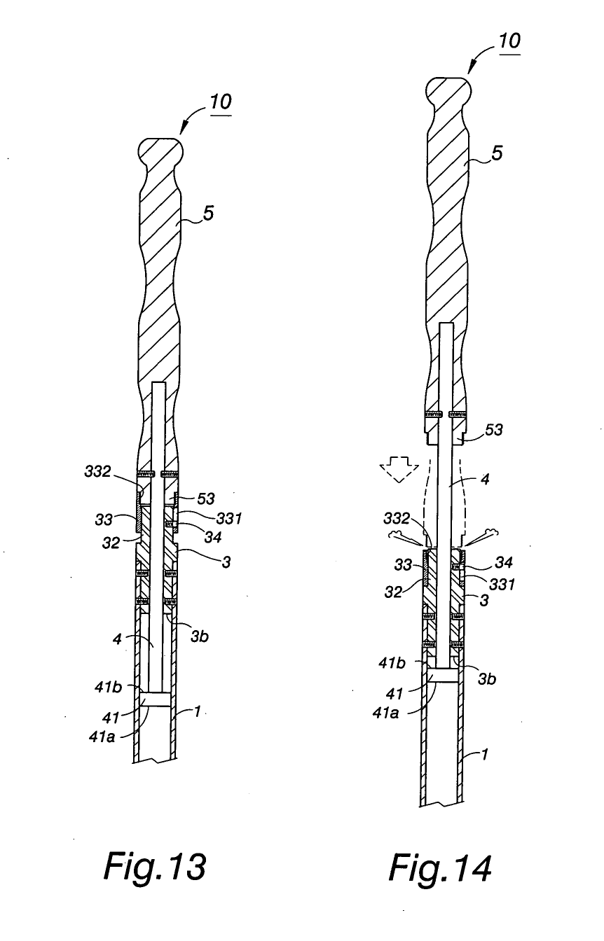

With reference to FIG. 13, a protrusion 32 extends from a second end (i.e. top boarder) of the stopper 3 of the tube body 1. The protrusion 32 can be engaged with a threaded sleeve 33 horizontally or laterally. The sleeve 33 has a long slot 331. A bolt 34 can be inserted into the slot 33 and then the thread sleeve 33 is fixed to the protrusion 32. Thereby, the moving length of the thread sleeve 22 in the protrusion 32 is confined and thus it is hard to separate from the protrusion. Moreover, a first end of the hammer 5 has exterior thread 53. When the thread sleeve 33 is moved upwards, the hammer 5 can be rotated to screw the exterior thread 52 into the thread sleeve 33, and then engaged with the interior thread of the thread sleeve 33. Thereby, the hammer 5 is screwed into the thread sleeve 33 securely without swift movement. Thus, in packaging and transferring the inserting rod 10, the hammer 5 will not be allowed to move to and fro reciprocally. Furthermore, the hammer 5 will keep in place securely, it may reduce the possible injury happened to thump fingers of ignorant children from dropping hammers off as the inserting rod 10 will not be in use. Referring to FIG. 14, an application of the inserting rod 10 in the third embodiment of the present invention is illustrated. When the hammer 5 is rotated to separate from the thread sleeve 33, the thread sleeve 33 will fall down by itself. The user may move the hammer 5 upward and downward to knock the protrusion 32 of the stopper 3. Then the tube body 1 and the inserting rod 2 will be withstood an axial force to lower down their positions. On the contrary, the inner hammer 41 knocks a first end of the stopper 3 repeatedly, and then the tube body 1 and the inserting rod 2 will be moved upwards.

What is claimed is:

1. A hammering inserting rod comprising:
a tube body;
an inserting rod secured to a bottom of the tube body; a bottom of the inserting rod being a pointed end;
a stopper secured to a top of the tube body; and a center of the stopper having a central bore;
a linkage passing through the central bore of the stopper; a first end of the linkage being installed with an inner hammer;
a hammer secured to a second end of the linkage; and
wherein a protrusion extends from a top of the stopper of the tube body; the protrusion is engaged with a thread sleeve; the thread sleeve has a long slot; a bolt is inserted into the slot and then the thread sleeve is fixed to the protrusion firmly;
by above components, a user holds the hammer to raise and lower the hammer repeatedly; the hammer will beat the stopper; or the inner hammer is used to beat the stopper so that the tube body and inserting head are inserted into soil or separates from ground.

2. The hammering inserting rod as claimed in claim 1, wherein the inserting rod is integrally formed with the tube body as a whole one.

3. The hammering inserting rod as claimed in claim 1, wherein a hole at the second end of the tube body can be decreased by forging or punching, can be substitute for passing through the linkage as a stopper with a central bore.

4. The hammering inserting rod as claimed in claim 1, wherein a second end of the stopper is an expanding protrusion rim.

5. The hammering inserting rod as claimed in claim 1, wherein an ear, lever actuated squeeze clamp, or a through hole is formed at an outer periphery of the tube body for binding on with a rope.
